(19) United States
(12) Patent Application Publication
(10) Pub. No.: US 2026/0034665 A1
(43) Pub. Date: Feb. 5, 2026

(54) METHOD AND SYSTEM OF POSITIONING ROBOTIC SURGICAL SYSTEM

(71) Applicant: BRAIN NAVI BIOTECHNOLOGY CO., LTD, Zhubei City (TW)

(72) Inventors: Chieh Hsiao Chen, Zhubei City (TW); Jung-Fu Hou, Zhubei City (TW); Yong-Lun Hsu, Zhubei City (TW)

(21) Appl. No.: 18/793,669

(22) Filed: Aug. 2, 2024

(51) Int. Cl. B25J 9/16; A61B 34/37; G06T 17/20; G06T 19/20

(52) U.S. Cl. CPC B25J 9/1605; A61B 34/37; B25J 9/1676; G06T 17/20; G06T 19/20; G06T 2210/21; G06T 2210/41; G06T 2219/2016

(57) ABSTRACT

Embodiments of the present invention relate to a method to position a robotic surgical system. The method includes generating a first three-dimensional (3D) model of the robotic surgical system, a second 3D model of a patient, and a planned surgical pathway. The first 3D model is represented by a set of polygon meshes but not by any volumetric mesh. The method includes selecting a first candidate position to position the first 3D model in a virtual environment, simulating movements or rotations of the first 3D model in the virtual environment; and determining whether a collision associated with the first 3D model occurs based on the simulated movements or rotations. The method includes generating status information of the first candidate position.

### BACKGROUND OF THE INVENTION

### Field of the Invention

Embodiments of the present invention relate generally to methods and systems of positioning a robotic surgical system in an operating room.

### Description of the Related Art

Unless otherwise indicated herein, the approaches described in this section are not prior art to the claims in this application and are not admitted to be prior art by inclusion in this section.

A robotic surgical system can provide precise controls for various complex surgical procedures. The robotic surgical system may include a control console and a patient cart. The control console and the patient cart may be integrated or separated from each other. The control console includes a control interface for a surgeon to control components of the robotic surgical system. By moving the patient cart, the robotic surgical system is configured to be positioned at a place adjacent to a surgical table, which is configured to support a patient in an operating room. The patient cart may also support a camera, a robotic arm assembly, and surgical instruments. The patient cart may be moveable so that the robotic surgical system can be positioned at different places in the operating room, depending on the requirements of different surgical procedures and/or different patients. Before a particular surgical procedure is performed for a particular patient, an efficient and accurate positioning of the robotic surgical system in the operating room is needed.

### BRIEF DESCRIPTION OF THE DRAWINGS

FIG. 1 is an illustrative figure showing an example robotic surgical system 100, arranged in accordance with some embodiments of the present disclosure;

FIG. 2A illustrates typical mesh 201 configured to represent a three-dimensional (3D) model of a patient cart of the robotic surgical system;

FIG. 2B illustrates typical mesh 202 configured to represent a 3D model of a shoulder of the robotic surgical system;

FIG. 2C illustrates typical mesh 203 configured to represent a 3D model of a first arm of the robotic surgical system;

FIG. 2D illustrates typical mesh 204 configured to represent a 3D model of an elbow of the robotic surgical system;

FIG. 2E illustrates typical mesh 205 configured to represent a 3D model of a second arm of the;

FIG. 2F illustrates typical mesh 206 configured to represent a 3D model of a wrist of the robotic surgical system;

FIG. 3 illustrates process 300 to generate a mesh representing a surface of a component of a robotic surgical system, arranged in accordance with some embodiments of the present disclosure;

FIG. 4A illustrates 3D model 410 of a first arm of a robotic surgical system, arranged in accordance with some embodiments of the present disclosure;

FIG. 4B illustrates primitive sphere mesh 420, arranged in accordance with some embodiments of the present disclosure;

FIG. 4C illustrates adjusted sphere mesh 430, arranged in accordance with some embodiments of the present disclosure;

FIG. 4D illustrates resampled mesh 440 that substantially matches a surface of a 3D model, arranged in accordance with some embodiments of the present disclosure;

FIG. 4E illustrates an enlarged perspective view 460 of a portion of a resampled mesh and an enlarged perspective view 470 of a simplified mesh corresponding to the same portion, arranged in accordance with some embodiments of the present disclosure;

FIG. 5A illustrates mesh 501 configured to represent a surface of a 3D model of a patient cart, arranged in accordance with some embodiments of the present disclosure;

FIG. 5B illustrates mesh 502 configured to represent a surface of a 3D model of a shoulder, arranged in accordance with some embodiments of the present disclosure;

FIG. 5C illustrates mesh 503 configured to represent a surface of a 3D model of a first arm 123, arranged in accordance with some embodiments of the present disclosure;

FIG. 5D illustrates mesh 504 configured to represent a surface of a 3D model of an elbow, arranged in accordance with some embodiments of the present disclosure;

FIG. 5E illustrates mesh 505 configured to represent a surface of a 3D model of a second arm, arranged in accordance with some embodiments of the present disclosure;

FIG. 5F illustrates mesh 506 configured to represent a surface of a 3D model of a wrist, arranged in accordance with some embodiments of the present disclosure;

FIG. 5G illustrates additional meshes 507, 508, and 509 configured to represent surfaces of 3D models associated with a robotic surgical system, arranged in accordance with some embodiments of the present disclosure;

FIG. 6 illustrates a process 600 to position a robotic surgical system, arranged in accordance with some embodiments of the present disclosure;

FIG. 7A illustrates virtual environment 700 including various loaded models, arranged in accordance with some embodiments of the present disclosure;

FIG. 7B illustrates an example user interface 701 of virtual environment 700, arranged in accordance with some embodiments of the present disclosure; and

FIG. 7C illustrates an example user interface 770 of virtual environment 700, arranged in accordance with some embodiments of the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated herein.

In the disclosure, the term “three-dimensional (3D) model” refers to a virtual object configured to represent a physical object using a collection of points distributed in a three-dimensional virtual environment, in which the points are connected by various geometric entities, such as triangles, lines or curved surfaces. The term “polygon mesh” refers to a type of mesh having a collection of vertices, edges, and faces that define a shape of a 3D model and also explicitly represent a surface of the 3D model. The term “volumetric mesh” refers to a type of mesh having a collection of vertices, edges, and faces that represents an interior volume of a 3D model. The term “primitive mesh” refers to a mesh configured to describe a basic shape to design and create a 3D model. In some embodiments, the “primitive mesh” in the disclosure may be a polygon mesh. The term “uniform faces” refers to faces having the same lengths and widths, and thus the faces are geometrically the same. The term “virtual environment” refers to a digital environment created to imitate operations of a physical robotic surgical system.

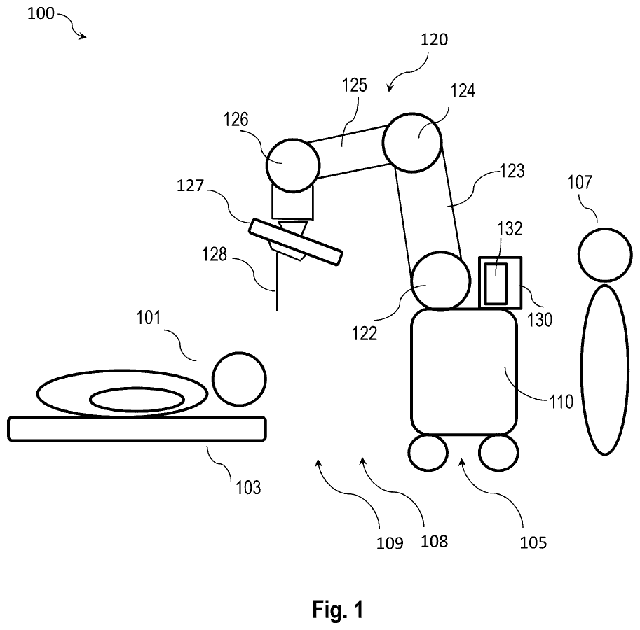

FIG. 1 is an illustrative figure showing an example robotic surgical system 100 configured to perform a surgical procedure on patient 101 on surgical table 103, arranged in accordance with some embodiments of the present disclosure. In some embodiments, robotic surgical system 100 includes patient cart 110, robotic arm assembly 120, and control console 130.

In some embodiments, patient cart 110 is configured to move so that robotic surgical system 100 is positioned at position 105 adjacent to surgical table 103 in an operating room. In addition, in some embodiments, patient cart 110 is configured to support robotic arm assembly 120 and control console 130.

In some embodiments, robotic arm assembly 120 includes, but not limited to, shoulder 122, first arm 123, elbow 124, second arm 125, wrist 126, terminal module 127, and surgical instrument 128. In some embodiments, terminal module 127 is an optical module including a camera. Terminal module 127 may be configured to capture one or more images of patient 101, and/or patient cart 110.

In some embodiments, control console 130 includes control interface 132. Control interface 132 is configured to interact with surgeon 107 so that surgeon 107 can control robotic arm assembly 120 to perform a surgical procedure on patient 101. More specifically, to perform the surgical procedure, surgeon 107 may interact with control interface 132 to control rotations of shoulder 122, elbow 124, and wrist 126 to actuate movements and/or rotations of first arm 123, second arm 125, terminal module 127, and surgical instrument 128 so that surgical instrument 128 moves along a planned surgical pathway of the surgical procedure into patient 101.

Conventionally, an operating room staff may position robotic surgical system 100 in the operating room at a position by moving patient cart 110 to the position based on his or her experience and also the type of surgical procedure to be performed on patient 101. For example, based on his or her experience with past patients, the staff may select position 105 to place robotic surgical system 100 for a deep brain stimulation procedure on patient 101, position 108 for a craniotomy procedure on patient 101, and position 109 for a biopsy procedure on patient 101.

However, such selections of positions may not be precise and may cause robotic surgical system 100 to be at a position from which the intended surgical procedure cannot be practically performed. For example, as the components of robotic arm assembly 120 move along the planned surgical pathway of the surgical procedure, an inappropriate positioning will cause undesirable collisions among the components of robotic surgical system 100, such as collisions between any two of patient cart 110, shoulder 122, first arm 123, elbow 124, second arm 125, wrist 126, terminal module 127, and surgical instrument 128. The surgical procedure then cannot be completed because of the collisions. Conventionally, patient cart 110 may be re-positioned to another position to address the collisions. However, re-positioning takes time and effort and does not guarantee that a different collision will not occur again. To address the technical problems above, methods and systems of appropriately positioning robotic surgical system 100 are described in more details below.

In some embodiments, a method to appropriately position a robotic surgical system includes performing simulations of movements and/or rotations of components of the robotic surgical system in a simulation or virtual environment. Such simulations may indicate whether collisions of components of the robotic surgical system will occur if a robotic arm assembly of the robotic surgical system is actuated to move along a planned surgical pathway of a surgical procedure to be performed on a patient.

In some embodiments, the simulations may be performed based on three-dimensional (3D) models of the components of the robotic surgical system. The 3D models may be represented by meshes.

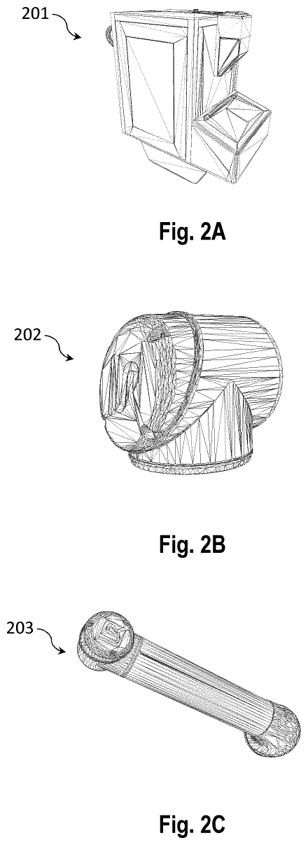

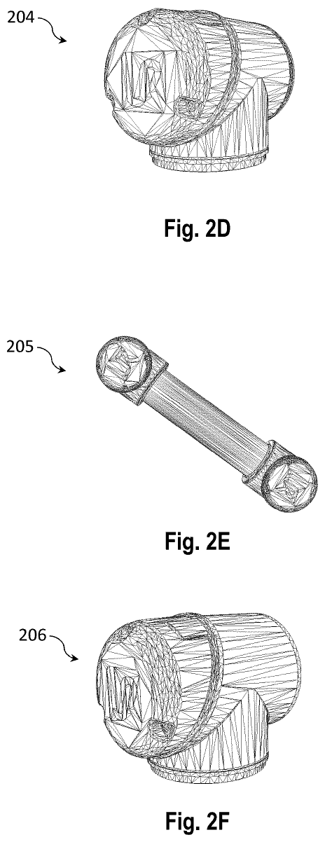

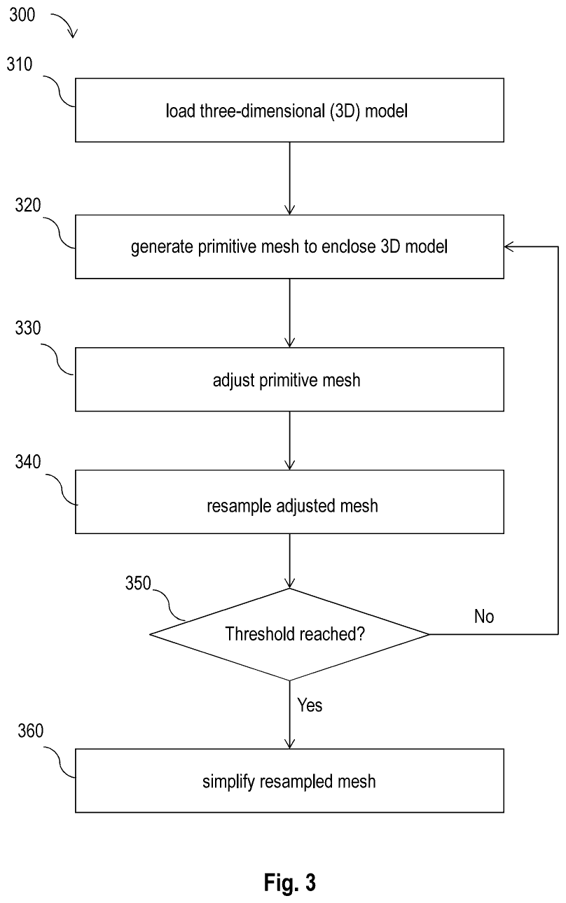

Each of FIG. 2A to 2F respectively represents a typical mesh defining a 3D model of a component of a robotic surgical system, such as robotic surgical system 100. More specifically, in conjunction with FIG. 1, FIG. 2A illustrates typical mesh 201 configured to represent a 3D model of patient cart 110; FIG. 2B illustrates typical mesh 202 configured to represent a 3D model of shoulder 122; FIG. 2C illustrates typical mesh 203 configured to represent a 3D model of first arm 123; FIG. 2D illustrates typical mesh 204 configured to represent a 3D model of elbow 124; FIG. 2E illustrates typical mesh 205 configured to represent a 3D model of second arm 125; and FIG. 2F illustrates typical mesh 206 configured to represent a 3D model of wrist 126. Typical meshes 201, 202, 203, 204, 205, and 206 may be obtained from the manufacturer of robotic surgical system 100. For example, mesh 201 may include a polygon mesh representing the surface of the 3D model of patient cart 110 and a volumetric mesh representing the interior volume of the 3D model of patient cart 110. Similarly, each of the other typical meshes 202, 203, 204, 205, and 206 may also include a polygon mesh and a volumetric mesh representing the surface and the interior volume of the 3D model of the respective component of the robotic surgical system.

However, there are challenges in performing simulations based on typical meshes 201, 202, 203, 204, 205, and 206. For example, the numbers of vertices, edges, and faces included in any of typical meshes 201, 202, 203, 204, 205, and 206 tend to be large to more precisely represent the 3D models of surfaces and interior volumes of patient cart 110, shoulder 122, first arm 123, elbow 124, second arm 125 and wrist 126, respectively. Such large numbers of vertices, edges, and faces may cause undesirable computation delays in simulations.

In addition to the large numbers of vertices, edges, and faces included in typical meshes 201, 202, 203, 204, 205, and 206, processing different types of vertices, edges, and faces included in typical meshes 201, 202, 203, 204, 205, and 206 in simulation may further lengthen the computation delays. For example, any of typical meshes 201, 202, 203, 204, 205, and 206 may include different sizes of triangles.

Moreover, to simulate collisions of the components of the robotic surgical system, volumetric meshes in the typical meshes 201, 202, 203, 204, 205, and 206 may not be required because collisions can be determined based on just the polygon meshes defining the surfaces of the components of the robotic surgical system.

To address the drawbacks of using typical meshes 201, 202, 203, 204, 205, and 206 in simulations, in some embodiments, FIG. 3 illustrates a process 300 to generate a mesh representing the surface of a component of a robotic surgical system. In some embodiments, simulating collisions of components of the robotic surgical system may be efficiently performed based on the generated mesh. Process 300 may include one or more operations, functions, or actions as illustrated by blocks 310, 320, 330, 340, 350 and/or 360, which may be performed by hardware, software and/or firmware. The various blocks are not intended to be limiting to the described embodiments. The outlined steps and operations are only provided as examples, and some of the steps and operations may be optional, combined into fewer steps and operations, or expanded into additional steps and operations without detracting from the essence of the disclosed embodiments. Although the blocks are illustrated in a sequential order, these blocks may also be performed in parallel, and/or in a different order than those described herein.

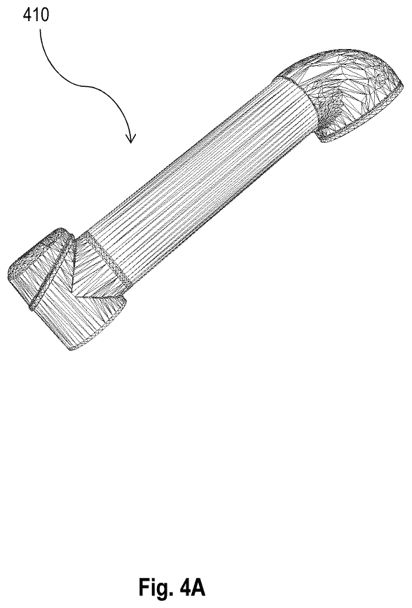

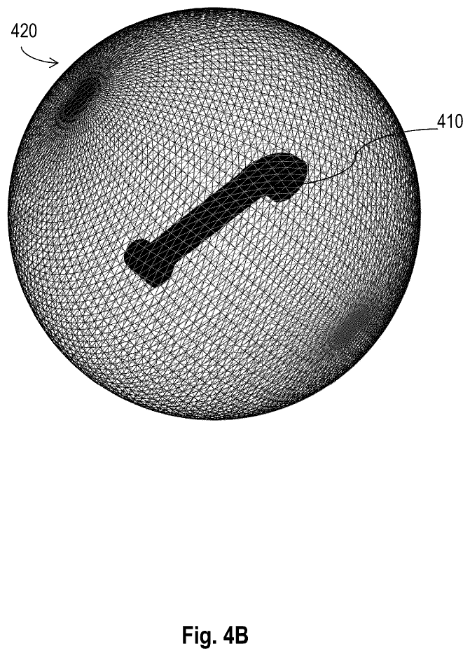

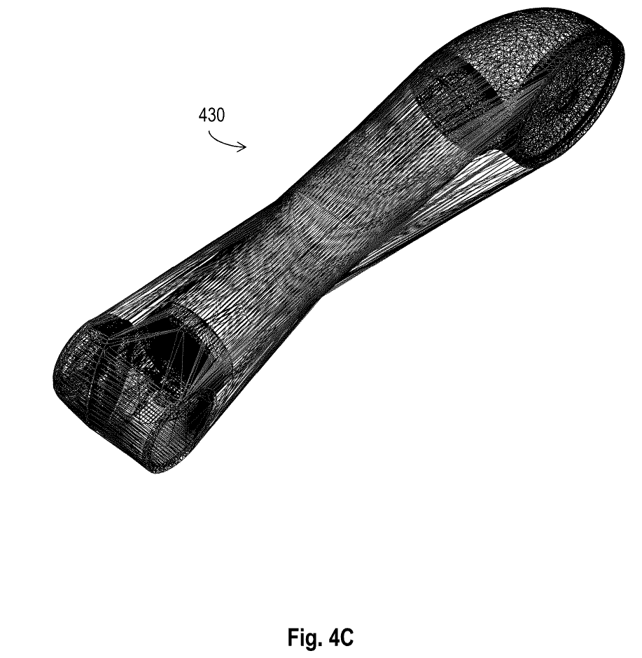

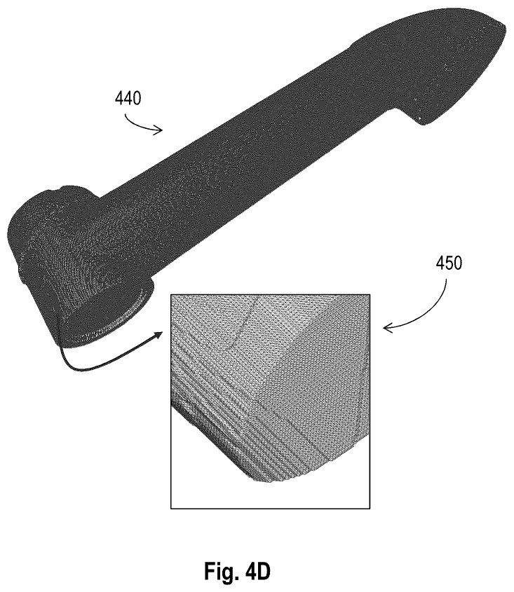

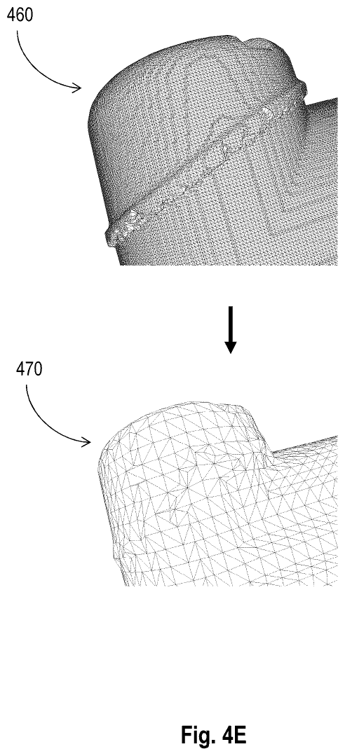

Process 300 may begin at block 310, “load three-dimensional (3D) model.” In conjunction with FIG. 1, FIG. 4A illustrates 3D model 410 of first arm 123. In some embodiments, 3D model 410 is provided by a manufacturer of first arm 123 and loaded in a virtual environment. In conjunction with FIG. 2C, 3D model 410 may be represented by typical mesh 203.

Block 310 may be followed by block 320, “generate primitive mesh to enclose 3D model.” In some embodiments, a primitive mesh is configured to enclose a 3D model. In other words, the primitive mesh is configured to surround or hold in the 3D model in a three-dimensional manner. In some embodiments, a primitive mesh is generated in the virtual environment to enclose the 3D model loaded in block 310. The primitive mesh may include a basic shape mesh. For example, some basic shape meshes include, but not limited to, a cube mesh, a sphere mesh, a cylinder mesh, a cone mesh, and a torus mesh. In some embodiments, FIG. 4B illustrates a primitive sphere mesh 420. In conjunction with FIG. 4B, in block 320, in some embodiments, sphere mesh 420 is generated to enclose the 3D model loaded in block 310. More specifically, the radius of sphere mesh 420 is set to be a value so that sphere mesh 420 is configured to be able to enclose the 3D model loaded in block 310. In some embodiments, sphere mesh 420 includes a standard collection of vertices, edges, and faces describing the surface of a sphere.

Block 320 may be followed by block 330, “adjust primitive mesh.” In some embodiments, the primitive mesh generated in block 320 is adjusted to match a surface of the 3D model loaded in block 310. In some embodiments, FIG. 4C illustrates adjusted sphere mesh 430. In conjunction with FIG. 4C, block 330 may include, but not limited to, operations of adjusting relationships among vertices, edges, and faces of sphere mesh 420 to generate adjusted sphere mesh 430 to match the surface of the 3D model loaded in block 310.

Block 330 may be followed by block 340, “resample adjusted mesh.” In some embodiments, the mesh adjusted in block 330 is resampled to generate a resampled mesh including uniform faces. In some embodiments, two uniform faces generally have the same shape because their respective lengths and widths are the same. In block 340, downsampling and upsampling may be used. Larger faces in the adjusted mesh may be upsampled and smaller faces in the adjusted mesh may be downsampled.

Block 340 may be followed by block 350, “threshold reached?” In some embodiments, the threshold may be a preset number of iterations of performing blocks 320, 330, and 340. Block 350 may be followed by block 320 in response to the number of iterations performing blocks 320, 330, and 340 failing to reach the threshold (e.g., less than the present number), in which the resampled mesh in block 340 is to be the primitive mesh in block 320. Block 350 may be followed by block 360, “simplify resampled mesh,” in response to the number of iterations of performing blocks 320, 330, and 340 reaching the threshold (e.g., equal to or greater than the preset number).

In some other embodiments, the threshold may be a preset average distance from points of the resampled mesh to points of the 3D model in block 350 “threshold reached?” Block 350 may be followed by block 320 in response to the average distance failing to reach the threshold (e.g., greater than the preset average distance), in which the resampled mesh in block 340 is to be the primitive mesh in block 320. Block 350 may be followed by block 360, “simplify resampled mesh,” in response to the average distance reaching the threshold (e.g., equal to or less than the preset average distance).

In some embodiments, through iterations of performing blocks 320, 330, and 340, a final resampled mesh may substantially match the surface of the 3D model generated in block 310. FIG. 4D illustrates resampled mesh 440 that substantially matches the surface of the 3D model generated in block 310. In some embodiments, resampled mesh 440 may include uniform faces. An enlarged perspective view 450 in FIG. 4D illustrates some example uniform faces of resampled mesh 440.

To further illustrate “simplify resampled mesh” in block 360, in some embodiments, FIG. 4E illustrates an enlarged perspective view 460 of a portion of resampled mesh 440 in FIG. 4D and an enlarged perspective view 470 of a simplified mesh corresponding to the same portion. In conjunction with FIG. 4D, it is worth noting that the operations performed in “adjust primitive mesh” in block 330 and “resample adjusted mesh” in block 340 may result in a high density of vertices and faces in resampled mesh 440, which may cause undesirable computation delays. Therefore, in some embodiments, in block 360, mesh simplification operations may be performed on resampled mesh 440. In some embodiments, the mesh simplification operations may include, without limitation, clustering decimation or quadric edge collapse decimation operations. For example, the mesh simplification operation may include grouping vertices into clusters and then collapsing these clusters into single representative vertices to reduce number of the uniform faces in resampled mesh 440. Accordingly, in some embodiments, the dense mesh illustrated in enlarged perspective view 460 may be simplified to a corresponding sparse mesh illustrated in enlarged perspective view 470. In some embodiments, the simplified and sparse mesh shown in enlarged perspective view 470 may include a polygon mesh representing the surface of the first 3D model generated in block 310 but exclude a volumetric mesh representing the interior volume of the first 3D model generated in block 310.

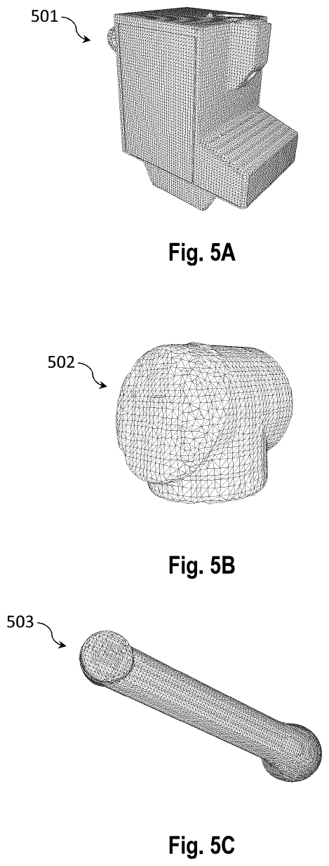

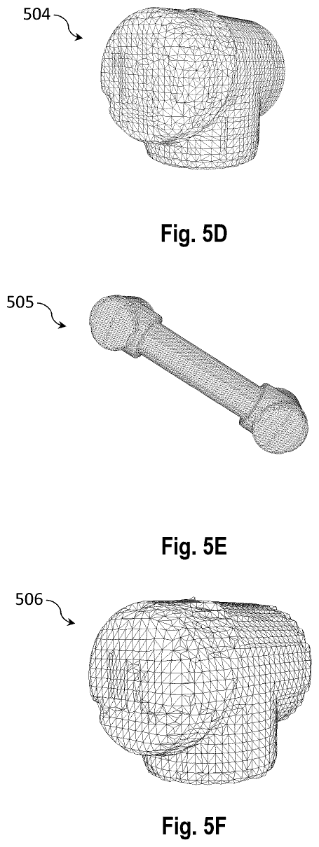

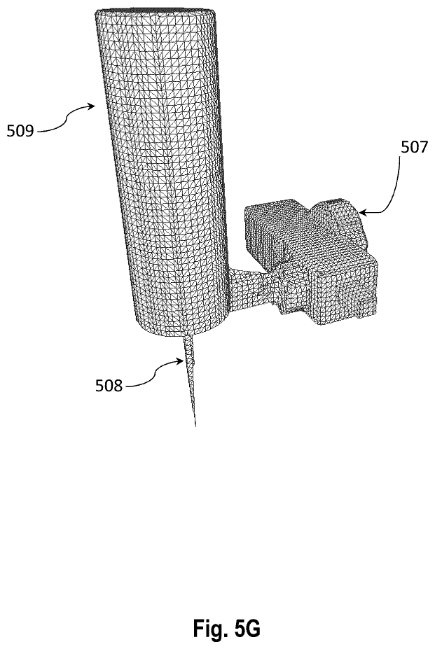

In some embodiments, similar meshes defining surfaces of other components of the robotic surgical system may also be generated based on process 300. In some embodiments, each of FIG. 5A to 5F respectively illustrates a mesh generated based on process 300 and represents a surface of a 3D model of a component of the robotic surgical system. Specifically, in conjunction with FIG. 1, FIG. 5A illustrates mesh 501 configured to represent a surface of a 3D model of patient cart 110; FIG. 5B illustrates mesh 502 configured to represent a surface of a 3D model of shoulder 122; FIG. 5C illustrates mesh 503 configured to represent a surface of a 3D model of first arm 123; FIG. 5D illustrates mesh 504 configured to represent a surface of a 3D model of elbow 124; FIG. 5E illustrates mesh 505 configured to represent a surface of a 3D model of second arm 125; and FIG. 5F illustrates mesh 506 configured to represent a surface of a 3D model of wrist 126.

Each of FIG. 5A to 5F respectively illustrates a mesh representing a surface of a 3D model of a component of the robotic surgical system but not representing an interior volume of the 3D model of the component of the robotic surgical system. Moreover, each of FIG. 5A to 5F respectively includes uniform faces. In contrast, the meshes illustrated in FIG. 2A to 2F include non-uniform faces. These non-uniform faces are different in shape.

In some embodiments, FIG. 5G illustrates additional meshes 507, 508, and 509 configured to represent surfaces of 3D models associated with the robotic surgical system. More specifically, in conjunction with FIG. 1, FIG. 5G illustrates mesh 507 configured to represent a surface of a 3D model of terminal module 127 and mesh 508 configured to represent a surface of a 3D model of surgical instrument 128. Meshes 507 and 508 are generated based on process 300 as described in details above.

In some embodiments, FIG. 5G further illustrates mesh 509. In some embodiments, mesh 509 is a cylinder polygon mesh. It is worth noting that mesh 509 does not correspond to any component of the robotic surgical system. Instead, mesh 509 may represent a working space for a surgeon to perform the surgical procedure. For example, the surgeon may need the working space to insert additional or replacement surgical instruments. In another example, the surgeon may need the working space to drill a hole through the skull of a patient. In some embodiments, mesh 509 may correspond to a physical working space in the operating room having a diameter between 50 mm to 200 mm and a length between 50 mm to 1000 mm. In some embodiments, in conjunction with FIG. 1, the working space may be positioned near (e.g., at the back of) surgical instrument 128 of robotic surgical system 100. In some embodiments, similar to meshes 501 to 508, mesh 509 also includes uniform faces. In some embodiments, the density of the uniform faces of mesh 509 may be similar to or the same as the density of the uniform faces of meshes 507 and 508.

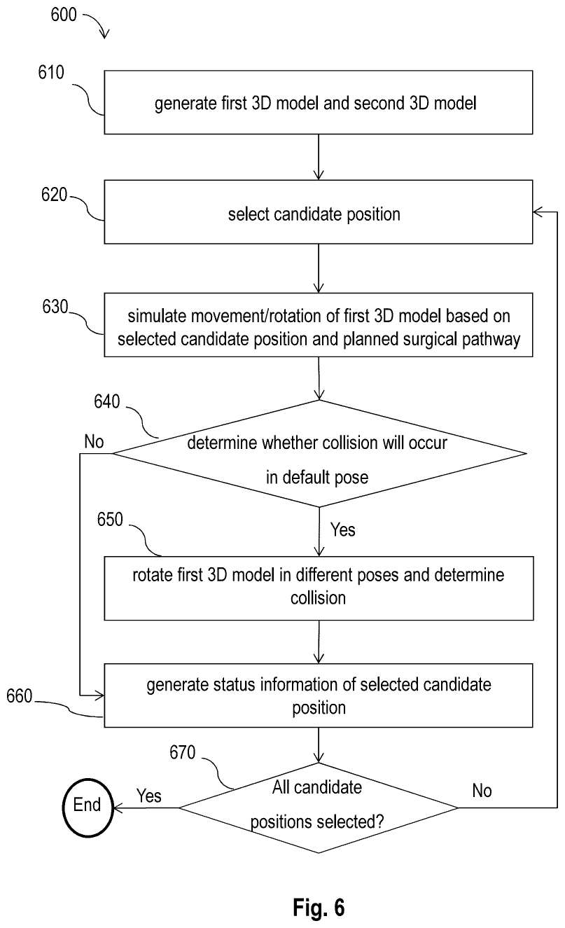

in some embodiments, FIG. 6 illustrates a process 600 to position a robotic surgical system. Process 600 may include one or more operations, functions, or actions as illustrated by blocks 610, 620, 630, 640, 650, 660, and/or 670, which may be performed by hardware, software and/or firmware. The various blocks are not intended to be limiting to the described embodiments. The outlined steps and operations are only provided as examples, and some of the steps and operations may be optional, combined into fewer steps and operations, or expanded into additional steps and operations without detracting from the essence of the disclosed embodiments. Although the blocks are illustrated in a sequential order, these blocks may also be performed in parallel, and/or in a different order than those described herein.

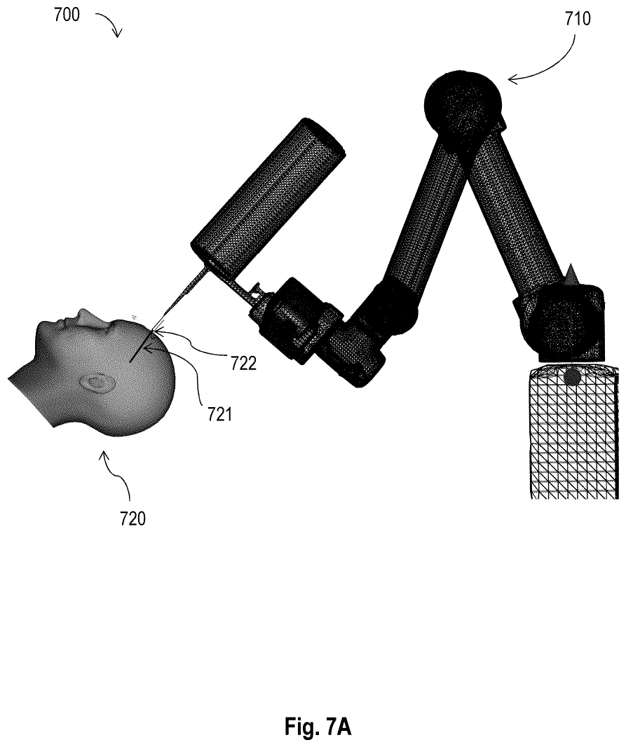

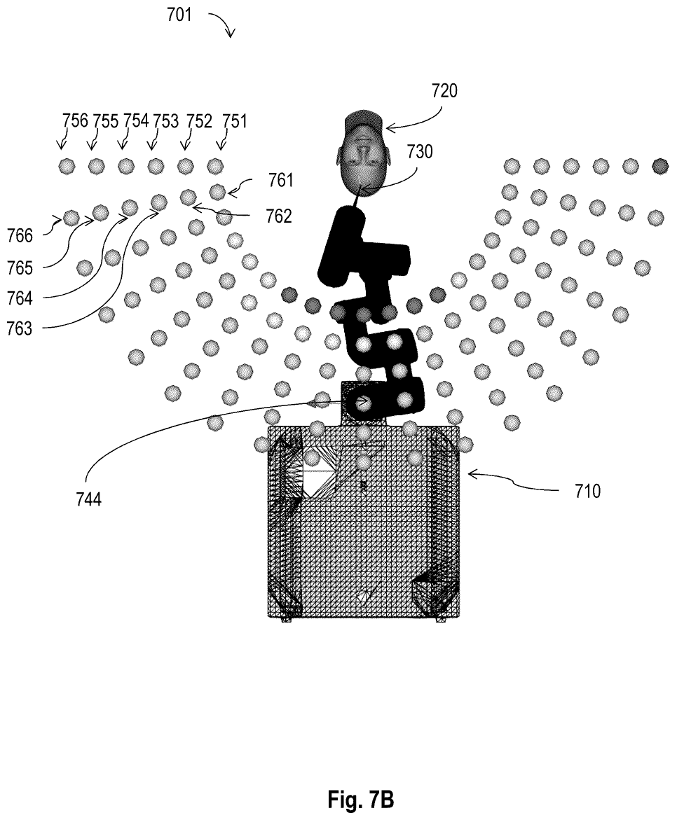

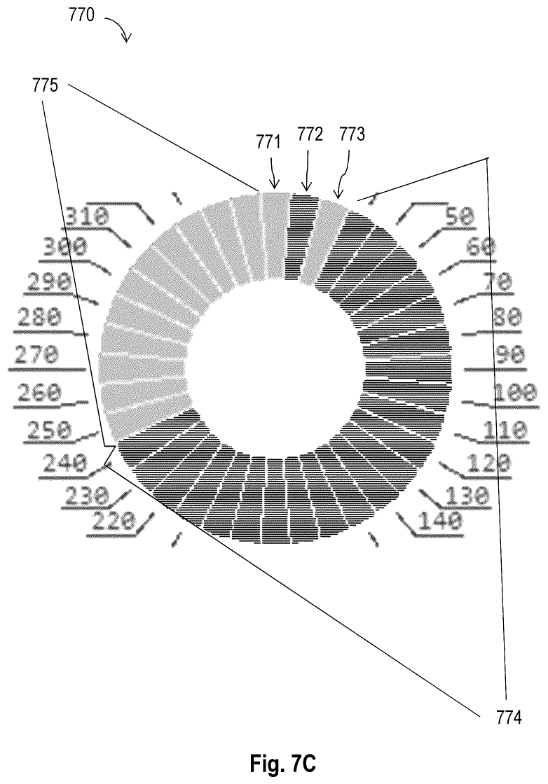

Process 600 may begin at block 610, “generate first 3D model and second 3D model.” In some embodiments, in conjunction with FIG. 5A to 5G, a first 3D model of the robotic surgical system is generated based on meshes 501, 502, 503, 504, 505, 506, 507, 508, and 509 in a virtual environment in block 610. In some embodiments, FIG. 7A illustrates virtual environment 700 including various 3D models. Specifically, robotic surgical system 3D model 710 associated with a set of meshes, such as meshes 501, 502, 503, 504, 505, 506, 507, 508, and 509, shown in FIG. 7A is generated in block 610.

In some embodiments, in block 610, a second 3D model of a patient and a planned surgical pathway is also generated. For example, before a surgical procedure is performed, some medical imaging techniques may be used to capture a snapshot of a patient's conditions, so that a surgical procedure plan may be formulated. The surgical procedure plan may include a planned surgical pathway. For example, the surgeon may order a medical image scan (e.g., CT or MRI) on a target region of the patient where the surgical procedure will be performed. Such a medical image scan may be performed a few days (e.g., 3 to 5 days) prior to the surgical procedure. A 3D model of the target region of the patient may be generated based on the medical image scan data using some known approaches. In some embodiments, in conjunction with FIG. 7A, a 3D model 720 of patient and planned surgical pathway 721 according to the formulated operation plan is generated in virtual environment 700 in block 610.

In some embodiments, block 610 may be followed by block 620, “select candidate position.” In some embodiments, FIG. 7B illustrates an example user interface 701 of virtual environment 700. In some embodiments, candidate position 744 is selected to position the robotic surgical system 3D model generated in block 610 in virtual environment 700. In some embodiments, candidate position 744 corresponds to a physical position of the physical robotic surgical system in the physical operating room. For illustration only, for example, candidate position 744 may correspond to a physical position of the physical robotic surgical system in an operating room that has a distance of 80 cm from the patient's head and 90 degrees from the patient's left ear and right ear.

In some embodiments, a plurality of candidate positions may be set and displayed in user interface 701 of virtual environment 700. For example, candidate positions may be set along a first degree from the patient's left ear but spaced from each other. As an illustration, in some embodiments, candidate position 751 may correspond to a physical position of the physical robotic surgical system in the operating room that has a distance of 50 cm from the patient's head and 0 degrees from the patient's left ear, candidate position 752 may correspond to a physical position of the physical robotic surgical system in the operating room that has a distance of 60 cm from the patient's head and 0 degrees from the patient's left ear, candidate position 753 may correspond to a physical position of the physical robotic surgical system in the operating room that has a distance of 70 cm from the patient's head and 0 degrees from the patient's left ear, candidate position 754 may correspond to a physical position of the physical robotic surgical system in the operating room that has a distance of 80 cm from the patient's head and 0 degrees from the patient's left ear, candidate position 755 may correspond to a physical position of the physical robotic surgical system in the operating room that has a distance of 90 cm from the patient's head and 0 degrees from the patient's left ear, and candidate position 756 may correspond to a physical position of the physical robotic surgical system in the operating room that has a distance of 100 cm from the patient's head and 0 degrees from the patient's left ear.

Similarly, in some embodiments, candidate positions may be set along a second degree from the patient's left ear but spaced from each other. As an illustration, in some embodiments, candidate position 761 may correspond to a physical position of the physical robotic surgical system in the operating room that has a distance of 50 cm from the patient's head and 10 degrees from the patient's left ear, candidate position 762 may correspond to a physical position of the physical robotic surgical system in the operating room that has a distance of 60 cm from the patient's head and 10 degrees from the patient's left ear, candidate position 763 may correspond to a physical position of the physical robotic surgical system in the operating room that has a distance of 70 cm from the patient's head and 10 degrees from the patient's left ear, candidate position 764 may correspond to a physical position of the physical robotic surgical system in the operating room that has a distance of 80 cm from the patient's head and 10 degrees from the patient's left ear, candidate position 765 may correspond to a physical position of the physical robotic surgical system in the operating room that has a distance of 90 cm from the patient's head and 10 degrees from the patient's left ear, and candidate position 766 may correspond to a physical position of the physical robotic surgical system in the operating room that has a distance of 100 cm from the patient's head and 10 degrees from the patient's left ear.

Similarly, in some embodiments, additional candidate positions may be set and displayed in user interface 701 as illustrated in FIG. 7B. In other embodiments, the candidate positions may be set in a relatively high density so that a heat map, instead of the discrete points as currently illustrated in FIG. 7B, is displayed in user interface 701. Although FIG. 7B shows different shades of grey in user interface 701, it should be apparent to one skilled in the art that colors can be used.

In some embodiments, block 620 may be followed by block 630, “simulate movement/rotation of first 3D model based on selected candidate position and planned surgical pathway.” In some embodiments, such simulations may be performed based on a kinematic modeling of the robotic surgical system. The kinematic modeling describes a relationship or conversion between (a) joint settings (e.g., joint angle configurations of the robotic surgical system) and kinematic settings (e.g., lengths of arms, the selected candidate position to be the origin of the robotic surgical system) associated with the robotic surgical system and (b) a pose of a surgical instrument of the robotic surgical system. That means, for a desired pose of the surgical instrument to reach the target region of the surgical procedure along the planned surgical pathway in virtual environment 700, each joint (e.g., meshes 502, 504, 506 in FIG. 5B, 5D, 5F, respectively) may rotate in such a way that the pose is reached. In conjunction with FIGS. 1, 5C, 5E, and 5G, rotations of meshes 502 (e.g., corresponding to shoulder 122), 504 (e.g., corresponding to elbow 124), 506 (e.g., corresponding to wrist 126) can actuate movements/rotations of meshes 503 (e.g., corresponding to first arm 123), 505 (e.g., corresponding to second arm 125), 507 (e.g., corresponding to terminal module 127), 508 (e.g., corresponding to surgical instrument 128), and 509 (e.g., corresponding to working space), respectively.

In some embodiments, block 630 may be followed by block 640, “determine whether collision will occur in default pose.” In some embodiments, in conjunction with FIGS. 5A, 5C, 5E, 5G, and 7B, a determination is made whether (1) any of meshes 503, 505, 507, 508, and 509 will collide with each other, (2) any of meshes 503, 505, 507, 508, and 509 will collide with mesh 501, or (3) any of meshes 503, 505, 507, 508, and 509 will collide with a patient's head at candidate position 744 selected in block 620 in response to mesh 508 being in a default pose. In some embodiments, the default pose may form a default angle between mesh 508 and mesh 507.

In some embodiments, collisions between any of meshes 501, 503, 505, 507, and 508 may refer to components of the physical robotic surgical system colliding with each other and the physical robotic surgical system not being able to reach the target region of the patient along the planned surgical pathway.

In some other embodiments, collisions between any of meshes 501, 503, and 505 with mesh 509 may refer to a physical working space for the surgeon to operate in being blocked.

In yet some other embodiments, collisions between any of meshes 501, 503, 505, 507, and 508 with the patient's head may refer to components of the physical robotic surgical system colliding with the patient's head.

In some embodiments, if no collision occurs between meshes in the default pose at candidate position 744, block 640 may be followed by block 660 “generate status information of selected candidate position.” Here, based on the determination in block 640, process 600 generates the status information of candidate position 744 in block 660. For example, in conjunction with FIG. 7B, generating status information may include, but not limited to, coloring candidate position 744 to be GREEN in user interface 701 to reflect that there is no collision between meshes in the default pose at candidate position 744. The GREEN indication is to remind the operating room staff or the surgeon that the physical robotic surgical system may be positioned at a physical position in the operating room corresponding to candidate position 744 in virtual environment 700, and surgical instrument 128 may be maintained in its default pose. In this scenario, to perform the surgical procedure, the physical robotic surgical system can be positioned in the operating room that has a distance of 80 cm from the patient's head and 90 degrees from the patient's left ear and right ear, and surgical instrument 128 may be maintained in its default pose.

On the other hand, if a collision occurs between the meshes in the default pose at candidate position 744, block 640 may be followed by block 650, “rotation first 3D model in different poses and determine collision.” Specifically, in block 650, process 600 further includes performing a round rotation, in which the default angle is set to be 0 degrees, of meshes defining robotic arm assembly 120 (i.e., meshes 502, 503, 504, 505, 506, 507, 508, and 509) around the axial direction of mesh 508 when a tip of mesh 508 is maintained at a point (e.g., entry point 722) on planned surgical pathway 721. At each of the different poses, another determination is made whether a collision between the meshes occurs. FIG. 7C illustrates an example user interface 770 of virtual environment 700. In some embodiments, user interface 770 is configured to display collision results between meshes in different poses in the round rotation. Similar to FIG. 7B, although FIG. 7C shows different shades of grey in user interface 770, it should be apparent to one skilled in the art that colors can be used.

In some embodiments, user interface 770 may include different regions 771, 772, 773, 774, and 775. Any of regions 771, 772, 773, 774, and 775 may include one or more slices. For example, region 771 may represent a collision result in the default pose. In some embodiments, region 771 may be colored RED in user interface 770 because block 640 has determined that there is a collision between meshes in the default pose at candidate position 744.

In some embodiments, in conjunction with FIGS. 1 and 5A to 5F, in block 650, meshes defining robotic arm assembly 120 (i.e., meshes 502, 503, 504, 505, 506, 507, 508, and 509) are rotated around the axial direction of mesh 508. In addition, in block 650, process 600 further includes determining whether there are collisions between meshes in different poses of mesh 508 at candidate position 744.

In some embodiments, region 772 may represent a collision result in a first pose of mesh 508 which is 10 degrees clockwise off from the default pose and region 773 may represent a collision result in a second pose of mesh 508 which is 20 degrees clockwise off from the default pose. For example, in response to no collisions between meshes in the first pose of mesh 508, region 772 may be colored GREEN in user interface 770. On the other hand, in response to one or more collisions between meshes in the second pose of mesh 508, region 773 may be colored RED in user interface 770. For illustrations only, region 774 may be colored GREEN to represent that no collisions between meshes would occur in poses corresponding to slices of region 774 and region 775 may be colored RED in user interface 770 to represent that one or more collisions between meshes would occur in poses corresponding to slices of region 775.

In some embodiments, block 650 may be followed by block 660, “generate status information of selected candidate position.” In one scenario, one or more collisions occur in the default pose (as determined in block 640) but no collisions occur in some poses at candidate position 744 (as determined in block 650), process 600 generates certain status information of candidate position 744 in block 660. For example, in conjunction with FIG. 7B, generating status information here may include, but not limited to, coloring candidate position 744 to be YELLOW in user interface 701. The YELLOW indication is to remind the operating room staff or the surgeon that the physical robotic surgical system may be positioned at a physical position in the operating room corresponding to candidate position 744 in virtual environment 700, and surgical instrument 128 may be maintained in a pose (e.g., the first pose discussed above) different from its default pose to perform the surgical procedure. In this scenario, to perform the surgical procedure, the physical robotic surgical system can be positioned in the operating room that has a distance of 80 cm from the patient's head and 90 degrees from the patient's left ear and right ear, and surgical instrument 128 may be maintained in a pose (e.g., any pose corresponding to a GREEN colored region in user interface 770) different from its default pose.

In another scenario, in response to one or more collisions occurring between meshes in all poses of mesh 508 at candidate position 744 (as determined in block 650), in conjunction with FIG. 7C, all regions 771, 772, 773, 774, and 775 are colored RED. Specifically, based on the determination made in block 650, generating status information in block 660 may include, but not limited to, coloring candidate position 744 to be RED in user interface 701 in FIG. 7B. The RED indication is to remind the operating room staff or the surgeon that the physical robotic surgical system cannot be positioned at a physical position in the operating room corresponding to candidate position 744 in virtual environment 700 to perform the surgical procedure. In this scenario, if the physical robotic surgical system is positioned in the operating room that has a distance of 80 cm from the patient's head and 90 degrees from the patient's left ear and right ear, the surgical procedure will not be able to be performed.

In some embodiments, block 660 may be followed by block 670, “all candidate position selected?” If all candidate positions have not been selected, block 670 may be looped back to block 620 to select a new candidate position (e.g., candidate position 751 in FIG. 7B) and blocks 630, 640, 650, and 660 will be repeated based on the new selected candidate position. If all candidate positions have been selected, process 600 will end in block 670.

The disclosure provides methods and systems to appropriately position a robotic surgical system in an operating room. Unlike conventional approaches that rely on staffs' experiences and often lead to re-positioning the robotic surgical system multiple times before a procedure can be performed, the disclosure provides an improved and efficient approach to position the robotic surgical system. In addition, the disclosure also provides a novel mesh generation approach so that many candidate positions in an operating room can be efficiently evaluated and considered.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers, as one or more programs running on one or more general-purpose or special-purpose programmable processors, as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of non-transitory computer-readable storage medium used to actually carry out the distribution. Examples of a computer-readable storage medium may include, but are not limited to, recordable/non-recordable media (e.g., read-only memory (ROM), random access memory (RAM), magnetic disk or optical storage media, flash memory devices, etc.).

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

We claim:

1. A method to position a robotic surgical system, comprising:
generating a first three-dimensional (3D) model associated with the robotic surgical system, a second 3D model associated with a patient, and a planned surgical pathway in a virtual environment, wherein the first 3D model is represented by a set of polygon meshes but not by any volumetric mesh;
selecting a first candidate position for the first 3D model in the virtual environment, wherein the first candidate position corresponds to a first physical candidate position of the robotic surgical system in a physical operating room;
simulating movements or rotations of the first 3D model in the virtual environment based on the first candidate position and the planned surgical pathway;
determining whether a collision associated with the first 3D model occurs in the virtual environment based on the simulated movements or rotations; and
generating status information of the first candidate position based on the determining.

2. The method of claim 1, further comprising:
generating the set of polygon meshes prior to generating the first 3D model, wherein the set of polygon meshes represents a surface of the first 3D model.

3. The method of claim 2, wherein generating the set of polygon meshes comprises:
loading a third 3D model associated with a component of the robotic surgical system, wherein a first polygon mesh is configured to represent a surface of the third 3D model and a first volumetric mesh is configured to represent an interior volume of the third 3D model; and
creating a primitive mesh to enclose the third 3D model.

4. The method of claim 3, wherein the primitive mesh is a sphere mesh.

5. The method of claim 3, wherein generating the set of polygon meshes further comprises:
adjusting the primitive mesh to match a surface of the component;
resampling the adjusted mesh; and
simplifying the resampled mesh.

6. The method of claim 5, wherein the resampled mesh includes first uniform faces having the first same shape.

7. The method of claim 3, wherein generating the set of polygon meshes further includes generating a second polygon mesh corresponding to a physical working space for a surgeon.

8. The method of claim 7, wherein the second polygon mesh is a cylindrical polygon mesh and includes second uniform faces having the second same shape.

9. The method of claim 1, wherein the determining further includes determining whether the collision occurs when the first 3D model is in a default pose.

10. The method of claim 9, in response to determining that the collusion occurs when the first 3D model is in a default pose, further comprising determining whether the collision occurs when the first 3D model is in another pose different from the default pose.

11. A system to position a robotic surgical system, comprising:
a processor; and
a non-transitory computer-readable storage medium containing a set of executable instructions which, in response to execution by the processor, cause the processor to:
generate a first three-dimensional (3D) model associated with the robotic surgical system and a second 3D model associated with a patient and a planned surgical pathway in a virtual environment, wherein the first 3D model is represented by a set of polygon meshes but not by any volumetric mesh;
select a first candidate position for the first 3D model in the virtual environment, wherein the first candidate position corresponds to a first physical candidate position of the robotic surgical system in a physical operating room;
simulate movements or rotations of the first 3D model in the virtual environment based on the first candidate position and the planned surgical pathway;
determine whether a collision associated with the first 3D model occurs in the virtual environment based on the simulated movements or rotations; and
generate status information of the first candidate position.

12. The system of claim 11, wherein the non-transitory computer-readable storage medium containing an additional set of executable instructions which, in response to execution by the processor, cause the processor to:
generate the set of polygon meshes prior to generating the first 3D model, wherein the set of polygon meshes represents a surface of the first 3D model.

13. The system of claim 12, wherein the non-transitory computer-readable storage medium containing an additional set of executable instructions which, in response to execution by the processor, cause the processor to:
load a third 3D model associated with a component of the robotic surgical system, wherein a first polygon mesh is configured to represent a surface of the third 3D model and a first volumetric mesh is configured to represent an interior volume of the third 3D model; and
create a primitive mesh to enclose the third 3D model.

14. The system of claim 13, wherein the primitive mesh is a sphere mesh.

15. The system of claim 13, wherein the non-transitory computer-readable storage medium containing an additional set of executable instructions which, in response to execution by the processor, cause the processor to:
adjust the primitive mesh to match a surface of the component;
resample the adjusted mesh; and
simplify the resampled mesh.

16. The system of claim 15, wherein the resampled mesh includes first uniform faces having the first same shape.

17. The system of claim 13, wherein the non-transitory computer-readable storage medium containing an additional set of executable instructions which, in response to execution by the processor, cause the processor to:
generate a second polygon mesh corresponding to a physical working space for a surgeon.

18. The system of claim 17, wherein the second polygon mesh is a cylindrical polygon mesh and includes second uniform faces having the second same shape.

19. The system of claim 11, wherein the non-transitory computer-readable storage medium containing an additional set of executable instructions which, in response to execution by the processor, cause the processor to:
determine whether the collision occurs when the first 3D model is in a default pose.

20. The system of claim 19, wherein the non-transitory computer-readable storage medium containing an additional set of executable instructions which, in response to execution by the processor, cause the processor to:
in response to determining that the collusion occurs when the first 3D model is in a default pose, determine whether the collision occurs when the first 3D model is in another pose different from the default pose.
